(19) United States
(12) Patent Application Publication
(10) Pub. No.: US 2016/0331872 A1
(43) Pub. Date: Nov. 17, 2016

(54) DESIGN AND ASSEMBLY OF GRADED-OXIDE TANTALUM POROUS FILMS FROM SIZE-SELECTED NANOPARTICLES AND DENTAL AND BIOMEDICAL IMPLANT APPLICATION THEREOF

(71) Applicant: OKINAWA INSTITUTE OF SCIENCE AND TECHNOLOGY SCHOOL CORPORATION, Okinawa (JP)

(72) Inventor: Mukhles Ibrahim SOWWAN, Okinawa (JP)

(73) Assignee: Okinawa Institute of Science and Technology School Corporation, Okinawa (JP)

(21) Appl. No.: 15/111,293

(22) PCT Filed: Jan. 15, 2015
(86) PCT No.: PCT/JP2015/000166

§ 371 Date: Jul. 13, 2016

Related U.S. Application Data
(60) Provisional application No. 61/928,321, filed on Jan. 16, 2014.

(51) Int. Cl. A61L 31/02; A61C 8/00; H01J 37/34; C23C 14/16; C23C 14/34; C23C 14/35; A61L 31/14; C23C 16/50

(52) U.S. Cl. CPC A61L 31/022; A61L 31/14; A61L 31/146; A61C 8/0015; C23C 16/50; C23C 14/165; C23C 14/3414; C23C 14/35; H01J 37/3405; H01J 37/3426; H01J 37/3423; A61L 2300/104; A61L 2300/404; A61L 2430/12; A61L 2400/12

(57) ABSTRACT

A porous film made of size-selected tantalum nanoparticles is formed on a substrate, the porous film having a graded oxidation profile perpendicular to a surface of the substrate.

### TECHNICAL FIELD

The present invention relates to designs and assembly of tantalum films and to their application to biomedical implants. This application hereby incorporates by reference U.S. Provisional Application No. 61/928,321, filed Jan. 16, 2014, in its entirety.

### BACKGROUND ART

Nanostructured films of either pure tantalum or its oxides exhibit many interesting properties, such as a wide band gap (Chaneliere et al. 1998), high photocatalytic activity under UV irradiation (Guo and Huang 2011), chemical resistance (Barr et al. 2006), high melting point (Stella et al. 2009), good mechanical strength (Chaneliere et al. 1998), and biocompatibility (Leng et al. 2006; Oh et al. 2011). These films have been widely utilized in memory devices (Lin et al. 1999), supercapacitors (Bartic et al. 2002), orthopedic instruments (Levine et al. 2006), photocatalysts (Goncalves et al. 2012), fuel cells (Seo et al. 2013) and X-ray contrast agents (Oh et al. 2011; Bonitatibus et al. 2012). Tantalum pentoxide (Ta₂O₅), the most thermodynamically stable of the tantalum oxides (Chaneliere et al. 1998), in particular, is well known for its desirable properties and numerous potential applications. It was first used in the 1970s as an antireflective layer for optical or photovoltaic applications, owing to its high refraction coefficient, low absorption, and high band gap (Balaji et al. 2002; El-Sayed and Birss 2009).

During the last two decades, with research on thin films receiving ever-increasing attention, Ta₂O₅ was also established as an excellent alternative to conventional dielectric films, such as SiO₂ and SiN, which were being pushed close to their physical limits in terms of thickness reduction and dielectric strength (Chaneliere et al. 1998; Alers et al. 2007).

Recently, Ta₂O₅ films have attracted additional interest from the research community due to their good biocompatibility and osteoconductivity properties (Leng et al. 2006; Levine et al. 2006), which make them strong candidates in the field of tissue engineering (Li et al. 2012). Nevertheless, for a material to be useful for biocompatible implants, it must act as a suitable substrate for cell culture and tissue regeneration. Flat metallic and metal-oxide implant scaffolds, although exhibiting biocompatible properties, generally do not support cell growth. To overcome this problem, surfaces of the potential implant materials need to be designed in a way that enables them to support the adhesion and organization of living cells (Levine et al. 2006; Han et al. 2011). Therefore, considering this promising application potential in biomedical industries, great efforts have been made to develop and further refine synthesis techniques for porous tantalum and tantalum oxide films. Unfortunately, controlled growth of such films is difficult and presents great challenges. Various fabrication techniques have been utilized with limited success, such as the sol-gel (Zhang et al. 1998), film sputtering (Cheng and Mao 2003), electrodeposition (Lee et al. 2004; Seo et al. 2013), gas-phase combustion (Barr et al. 2006), arc source deposition (Leng et al. 2006), e-beam evaporation (Stella et al. 2009; Bartic et al. 2002) and chemical vapor deposition (Seman et al. 2007).

### CITATION LIST

### Non Patent Literature

NPL 1: Alers G B, Werder D J, Chabal Y, Lu H C, Gusev E P, Garfunkel E, Gustafsson T, Urdahl R S (2007) Intermixing at the tantalum oxide/silicon interface in gate dielectric structures. Appl Phys Lett 73:1517-1519
NPL 2: Arcidiacono S, Bieri N R, Poulikakos D, Grigoropoulos C P (2004) On the coalescence of gold nanoparticles. Int. J. Multiph. Flow 30:979-994
NPL 3: Atanassova E, Tyuliev G, Paskaleva A, Spassov D, Kostov K (2004) XPS study of N2 annealing effect on thermal Ta2O5 layers on Si. Appl Surf Sci 225:86-99
NPL 4: Balaji T, Govindaiah R, Sharma M K, Purushotham Y, Kumar A, Prakash T L (2002) Sintering and electrical properties of tantalum anodes for capacitor applications. Mater Lett 56:560-563
NPL 5: Barr J L, Axelbaum R L, Macias M E (2006) Processing salt-encapsulated tantalum nanoparticles for high purity, ultra high surface area applications. J Nanopart Res 8:11-22.
NPL 6: Bartic C, Jansen H, Campitelli A, Borghs S (2002) Ta2O5 as gate dielectric material for low-voltage organic thin-film transistors. Organic Electronics 3:65-72
NPL 7: Bonitatibus P J, Tones A S, Kandapallil B, Lee B D, Goddard G D, Colborn R E, Marino M E (2012) Preclinical Assessment of a Zwitterionic Tantalum Oxide Nanoparticle X-ray Contrast Agent. ACS Nano 6:6650-6658
NPL 8: Chaneliere C, Autran J L, Devine R A B, Balland B (1998) Tantalum pentoxide (Ta2O5) thin films for advanced dielectric applications. Materials Science and Engineering R: Reports 22:269-322
NPL 9: Chang J P, Opila R L, Alers G B, Steigerwald M L, Lu H C, Garfunkel E, Gustafsson T (1999) Interfacial reaction and thermal stability of Ta1O5/TiN for metal electrode capacitors. Solid State Technol 42:43-48
NPL 10: Cheng H, Mao C (2003) The effect of substrate temperature on the physical properties of tantalum oxide thin films grown by reactive radio-frequency sputtering. Mater. Res. Bulletin 38:1841-1849
NPL 11: Das B, Banerjee A (2007) Implementation of complex nanosystems using a versatile ultrahigh vacuum nonlithographic technique. Nanotechnology 18:445202
NPL 12: Ding F, Rosen A, Bolton K (2004) Size dependence of the coalescence and melting of iron clusters: A molecular-dynamics study. Phys Rev B 70:075416
NPL 13: Eggersdorfer M L, Kadau D, Herrmann H J, Pratsinis S E (2012) Aggregate morphology evolution by sintering: Number and diameter of primary particles. J Aerosol Sci 46:7-19
NPL 14: El-Sayed H A, Birss V I (2009) Controlled Interconversion of Nanoarray of Ta Dimples and High Aspect Ratio Ta Oxide Nanotubes. Nano Lett 9:1350-1355
NPL 15: Finnis M W, Sinclair J E (1984) A simple empirical N-body potential for transition metals. Phil Mag A 50:45-55
NPL 16: Gale J D (1997) GULP: A computer program for the symmetry-adapted simulation of solids. J Chem Soc, Faraday Trans 93:629-637
NPL 17: Goncalves R V, Migowski P, Wender H, Eberhardt D, Weibel D E, Sonaglio F C, Zapata M J M, Dupont J, Feil A F, Teixeira S R (2012) Ta2O5 Nanotubes Obtained by Anodization: Effect of Thermal Treatment on the Photocatalytic Activity for Hydrogen Production. J Phys Chem C 116:14022-14030
NPL 18: Guo G, Huang J (2011) Preparation of mesoporous tantalum oxide and its enhanced photocatalytic activity. Mater Lett 65: 64-66.
NPL 19: Han Y, Zhou J H, Zhang L, Xu K W (2011) A multi-scaled hybrid orthopedic implant: bone ECM-shaped Sr-HA nanofibers on the microporous walls of a macroporous titanium scaffold. Nanotechnology 22:275603
NPL 20: Hollaway P H, Nelson G S (1979) Preferential sputtering of Ta2O5 by argon ions. J Vac Sci Technol 16:793-796
NPL 21: Moo J G S, Awaludin Z, Okajima T, Ohsaka T (2013) An XPS depth-profile study on electrochemically deposited TaOx. J Solid State Electrochem 17:3115-3123.
NPL 22: Kart H H, Wang G, Karaman I, Cagin T (2009) Molecular dynamics study of the coalescence of equal and unequal sized Cu nanoparticles. Int J Mod Phys C 20:179-196
NPL 23: Kerrec O, Devilliers D, Groult H, Marcus P (1998) Study of dry and electro-generated Ta2O5 and Ta/Ta2O5/Pt structures by XPS. Mat Sci Eng B 55:134-142
NPL 24: Leng Y X, Chen J Y, Yang P, Sun H, Wang J, Huang N (2006) The biocompatibility of the tantalum and tantalum oxide films synthesized by pulse metal vacuum arc source deposition. Nucl Instrum Meth B 242:30-32
NPL 25: Levine B R, Sporer S, Poggie R A, Valle C J D, Jacobs J J (2006) Experimental and clinical performance of porous tantalum in orthopedic surgery. Biomaterials 27:4671-4681
NPL 26: Lewis L J, Jensen P, Barrat J L (1997) Melting, freezing, and coalescence of gold nanoclusters. Phys Rev B 56:2248-2257
NPL 27: Li Y, Zhang S, Guo L, Dong M, Liu B, Mamdouh W (2012) Collagen coated tantalum substrate for cell proliferation. Colloids and Surfaces B: Biointerfaces 95:10-15
NPL 28: Lin J, Masaaki N, Tsukune A, Yamada M (1999) Ta2O5 thin films with exceptionally high dielectric constant. Appl Phys Lett 74:2370
NPL 29: Oh M H, Lee N, Kim H, Park S P, Piao Y, Lee J, Jun S W, Moon W K, Choi S H, Hyeon T (2011) Large-scale synthesis of bioinert tantalum oxide nanoparticles for X-ray computed tomography imaging and bimodal image-guided sentinel lymph node mapping. J Am Chem Soc 133:5508-5515
NPL 30: Palmer R E, Pratontep S, Boyen H G (2003) Nanostructured surfaces from size-selected clusters. Nature Mater 2:443-448
NPL 31: Popoka V N, Barke I, Campbell E E B, Meiwes-Broer K H (2011) Cluster-surface interaction: From soft landing to implantation. Surf Sci Rep 66:347-377
NPL 32: Seman M, Robbins J J, Agarwal S, Wolden C A (2007) Self-limiting growth of tantalum oxide thin films by pulsed plasma-enhanced chemical vapor deposition. Appl Phys Lett 90:131504
NPL 33: Seo J, Zhao L, Cha D, Takanabe K, Katayama M, Kubota J, Domen K (2013) Highly Dispersed TaOx Nanoparticles Prepared by Electrodeposition as Oxygen Reduction Electrocatalysts for Polymer Electrolyte Fuel Cells. J Phys Chem C 117:11635-11646
NPL 34: Singh V, Cassidy C, Bohra M, Galea A, Hawash Z, Sowwan M (2013) Surface morphology of films grown by size-selected Ta nanoparticles. Adv Mater Res 647:732-737
NPL 35: Stella K, Burstel D, Franzka S, Posth O, Diesing D (2009) Preparation and properties of thin amorphous tantalum films formed by small e-beam evaporators. J Phys D: Appl Phys 42:135417
NPL 36: Zhang J Y, Bie L J, Dusastre V, Boyd I W (1998) Thin tantalum oxide films prepared by 172 nm Excimer lamp irradiation using sol-gel method. Thin solid films 318:252-256
NPL 37: Zhao S J, Wang S Q, Ye H Q (2001) Coalescence of three silver nanoclusters: a molecular dynamics study. J Phys: Condens Matter 13:8061-8069
NPL 38: Zhu H, Averback R S (1996) Sintering processes of two nanoparticles: A study by molecular dynamics simulations. Phil Mag Lett 73:27-33

### SUMMARY OF INVENTION

### Technical Problem

The above-mentioned various techniques have produced only limited success. Furthermore, there has been an increasing demand for dental and biomedical implants that are easily installed and maintained.

Accordingly, the present invention is directed to designs and assembly of graded-oxide tantalum porous films and to their application to dental and biomedical implants.

An object of the present invention is to provide designs and assembly of graded-oxide tantalum porous films in a reasonably inexpensive, well-controlled manner.

Another object of the present invention is to provide designs and assembly of graded-oxide tantalum porous films composed of size-selected nanoparticles.

Another object of the present invention is to provide dental or biomedical implants that are initially hydrophilic, but becomes hydrophobic soon thereafter.

### Solution to Problem

To achieve these and other advantages and in accordance with the purpose of the present invention, as embodied and broadly described, in one aspect, the present invention provides a porous film made of size-selected tantalum nanoparticles, formed on a substrate, the porous film having a graded oxidation profile perpendicular to a surface of the substrate.

In another aspect, the present invention provides a dental implant comprising an implant base and a coating on the implant base, wherein the coating is made of a porous film made of size-selected tantalum nanoparticles, formed on the implant base, the porous film having a graded oxidation profile perpendicular to a surface of the implant base.

In the porous film made of size-selected tantalum nanoparticles described above, oxidation of the tantalum nanoparticles may be higher on a top surface of the film and may be progressively lower towards a bottom surface of the film that is on the substrate.

The porous film made of size-selected tantalum nanoparticles described above may further include a mono-disperse layer of silver (Ag) deposited on the porous film, thereby providing enhanced antimicrobial properties.

In the dental implant described above, oxidation of the tantalum nanoparticles in the porous film may be made higher on a top surface of the film and may be progressively lower towards a bottom surface of the film that is on the implant base.

The dental implant describe above may further include a mono-disperse layer of silver (Ag) deposited on the porous film, thereby providing enhanced antimicrobial properties.

In the dental implant described above, the implant base may be made of a Ti alloy or tungsten alloy.

In another aspect, the present invention provides a biomedical implant comprising an implant base; and a porous film made of size-selected tantalum nanoparticles, formed on the implant base, the porous film having a graded oxidation profile perpendicular to a surface of the implant base.

### Advantageous Effects of Invention

According to one or more aspects of the present invention, it becomes possible to provide porous films with a graded oxidation profile perpendicular to the substrate surface, using size-selected tantalum nanoparticle deposition in a controlled and/or efficient manner, which allows for surface manipulation and design of nanoporous films for various biomedical and technological applications. Further, when applied to dental or biomedical implants, the present invention provides a dental/biomedical implant that is hydrophilic initially and becomes hydrophobic soon thereafter, thereby providing very convenient and advantageous dental/biomedical implants in dental and biomedical industries.

Additional or separate features and advantages of the invention will be set forth in the descriptions that follow and in part will be apparent from the description, or may be learned by practice of the invention. The objectives and other advantages of the invention will be realized and attained by the structure particularly pointed out in the written description and claims thereof as well as the appended drawings.

It is to be understood that both the foregoing general description and the following detailed description are exemplary and explanatory, and are intended to provide further explanation of the invention as claimed.

### BRIEF DESCRIPTION OF DRAWINGS

FIG. 1 is a schematic diagram of the magnetron-sputter inert-gas condensation set-up used for growth of tantalum nanoparticles and porous films according to an embodiment of the present invention.

FIG. 2 shows average particle sizes as a function of deposition parameters for nanoparticles for Ar flow at fixed aggregation length of 125 mm and for aggregations lengths at fixed Ar flow of 30 standard cubic centimeters per minute at constant DC magnetron power on 54 W.

FIG. 3 is (a) AFM topography image of a sample with low tantalum nanoparticle coverage deposited onto a silicon substrate with (b) height histogram. A Gaussian fit to the histogram is shown in the solid line. The average height is 3.8 nm, in good agreement with the pre-selected size of 3.0 nm by the QMF.

FIG. 4 shows (a) Bright-field TEM and (b) high-angle-annular dark-field STEM micrographs of tantalum/tantalum oxide nanoparticles directly deposited on a silicon nitride membrane. The insets show high-magnification image, where particles are amorphous in nature (inset in (a)) and a core-shell structure consisting of a metallic tantalum core covered with tantalum oxide (inset in (b)).

FIG. 5 shows the measured EDS spectra of tantalum/tantalum oxide nanoparticles and between nanoparticles. The EDS spectra indicate that the nanoparticle area (marked by numeral 2) contains Ta and O, as expected.

FIG. 6 show examples of characteristic resultant aggregates after a 100 ps molecular dynamics run for two and three nanoparticle configurations at temperature range 100 K to 2300 K for systems consisting of 2 or 3 nanoparticles.

FIG. 7 shows AFM surface morphology of (a) non-oxidized high coverage tantalum nanoparticles deposited onto a silicon substrate and (b) oxidized high coverage tantalum nanoparticles deposited onto a silicon substrate. The respective insets show a high-magnification image, which shows that the roughness increased after oxidation of Ta nanoparticles.

FIG. 8 shows an SEM image of high coverage tantalum nanoparticles deposited onto a silicon substrate. The inset shows a high-magnification image, where the porous nature of the film with elongated coalesced nanoparticles and pores can be observed.

FIG. 9 shows the observed grazing angle X-ray diffraction pattern of a nanoporous film on a silicon substrate at a fixed grazing angle of 0.5°. No peaks given for tantalum and tantalum oxide phases can be observed, except for a broad diffuse peak, which is typically the signature of amorphous nanoparticle films.

FIG. 10 shows XPS study: (a) survey spectra with the inset showing the fitted spectra of Ta 4f core level at the surface, and (b) a sequence of depth profile spectra shown with etching time with the inset showing the binding energy difference for the first and last spectra of Ta (4f_{7/2}). First spectra and last spectra are before etching and after etching for 420 s. Results indicate a graded oxidation profile perpendicular to the substrate.

FIG. 11 is a schematic illustration of porous tantalum film with a graded oxidation profile perpendicular to the surface of the substrate. Larger pore sizes near the surface allow for oxidation of tantalum to tantalum oxide. Oxidation levels decrease deeper into the film, leading to pure metallic tantalum near the film/substrate interface.

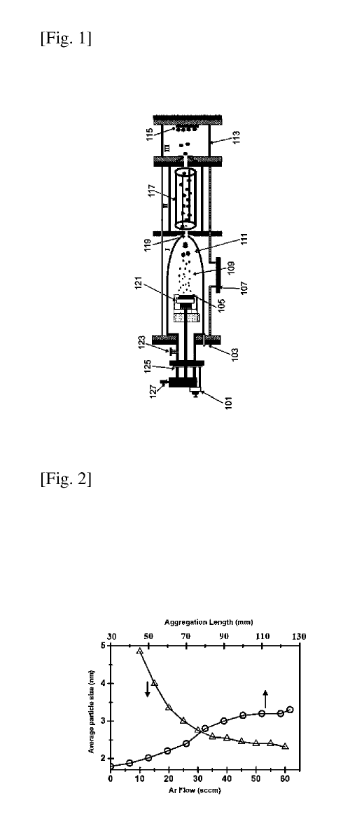

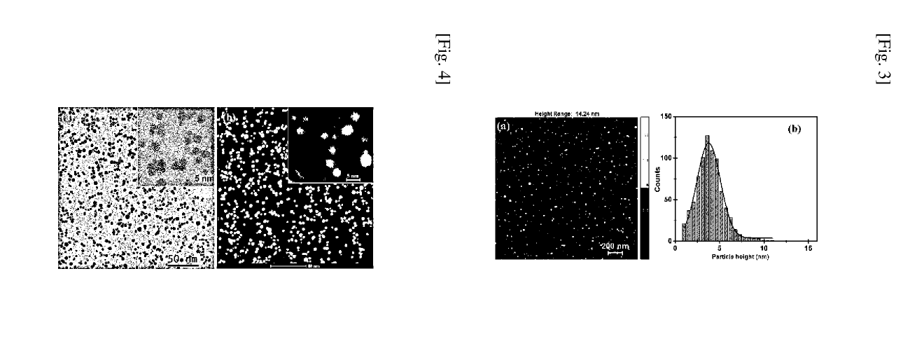

### DESCRIPTION OF EMBODIMENTS

The present inventor utilized a magnetron-sputter inert-gas aggregation system to fabricate customized porous films with a graded oxidation profile perpendicular to the substrate, assembled from discretely-deposited, size-selected, tantalum nanoparticles. This approach is relatively inexpensive, versatile, reproducible, and integrates all the steps for porous film growth into a continuous, well-controlled process (Palmer et al. 2003; Das and Banerjee 2007). Molecular dynamics (MD) computer simulations were employed to enhance understanding of nanoparticle coalescence during film growth, which largely affects the porosity of the film. Aberration-corrected scanning transmission electron microscopy (STEM), high-resolution transmission electron microscopy (HRTEM), atomic force microscopy (AFM), scanning electron microscopy (SEM), and grazing incidence x-ray diffraction (GIXRD) were used to study the morphology and structure of the tantalum nanoparticles and porous films. X-ray photo-electron spectroscopy (XPS) with depth profile analysis was applied to reveal oxidation states perpendicular to the surface of the substrate.

A tantalum magnetron-sputter target (purity >99.95%) with dimensions of 25 mm diameter×3 mm thick was purchased from Kurt J. Lesker (PA, USA). As substrates for AFM, SEM, XPS and GIXRD measurements, silicon dice/wafers with (100) orientation were purchased from MTI Corporation (CA, USA). Silicon dice/wafers were ultra-sonicated in acetone, 2-propanol and deionized purified water (5 minutes each), and subsequently dried in a stream of high purity nitrogen before placement in the vacuum chamber. Cleaned silicon dice surfaces exhibited typical root mean square (rms) roughness of 0.2 nm. Silicon nitride (Si₃N₄) membranes (200 nm thick) were purchased from Ted Pella Inc. (CA, USA), as substrates for TEM analysis.

An ultra-High Vacuum (UHV) based gas-phase nanoparticles deposition system (from Mantis Deposition Ltd, UK) was used for fabrication of the tantalum porous films of the present invention. FIG. 1 is a schematic diagram of the magnetron-sputter inert-gas condensation set-up used for growth of tantalum nanoparticles and porous films according to an embodiment of the present invention. Nanoparticles are formed in the aggregation zone 111 (labeled-I), then size-selected using QMF 117 (labeled-II), and deposited on a substrate 115 in the deposition chamber 113 (labeled-III). Major components of the deposition system are the aggregation zone 111, quadruple mass filter (QMF) 117, and substrate chamber 113 (FIG. 1). The aggregation zone 111 contains a sputtering magnetron head 121, capable of housing multiple sputter targets 105 (25 mm in diameter). Argon (Ar) is injected into the aggregation zone 111 as the sputtering gas at the magnetron head 121. Differential pumping through a small exit aperture 119 (5 mm diameter) leads to development of relatively high pressure inside the aggregation zone 111, resulting in coalescence of sputtered atoms and subsequent cluster growth. The walls of the aggregation zone form an enclosing water-cooled jacket, with a constant flow of water at 279 K. The aggregation zone length can be adjusted by translating the position of the magnetron head using a linear positioning drive, from 30 mm (fully inserted) to 125 mm (fully retracted). The large pressure-differential on either side of the aperture leads to acceleration of nascent clusters from the (high-pressure) aggregation zone 111 towards the (low-pressure) deposition chamber 113.

<Nanoparticle Growth and Deposition Procedure>

Primary tantalum nanoparticles were formed by gas-phase condensation inside the aggregation zone 111 (Singh et al. 2013). Atomic metal vapor of tantalum 109 was produced from a tantalum target using a DC magnetron sputtering process as shown in FIG. 1. According to well-established growth models (Palmer et al. 2003), tantalum atoms subsequently lost their original kinetic energy through successive interatomic collisions with the inert Ar atoms in the gas-aggregation region, leading by aggregation to tantalum nanoparticles. The gas flow, pressure, magnetron power, and aggregation zone length were key parameters that could be conveniently adjusted to directly influence the nucleation process (Das and Banerjee 2007). Optimal process conditions for yield and size distribution were first explored, via in situ mass spectrum feedback and ex situ AFM studies.

The apparatus also include various other components: such as a linear drive 101 to move the DC magnetron 121; a connection for coolant water 103; a turbo pump port 107; a pressure gauge 123; an aggregation gas feed 125; and connections 127 for DC power and gas, as shown in FIG. 1.

The particle diameter was investigated for several sets of deposition parameters. FIG.

2 shows average particle sizes as a function of deposition parameters. The conditions used in this disclosure were: Ar flow rates of 30 standard cubic centimeters per minute (resulting in an aggregation zone pressure reading of 1.0×10⁻¹ mbar), DC magnetron power of 54 W, and aggregation zone length of the maximum value (125 mm). These conditions were used for all tantalum nanoparticles fabricated in this disclosure. The presence of unwanted species or contaminants was controlled by achieving good pre-deposition base pressures (about 1.5×10⁻⁶ mbar in the aggregation zone and about 8.0×10⁻⁸ mbar in the sample deposition chamber), utilization of high purity target, and verification of system cleanliness via in situ residual gas analyzer (RGA).

After the aggregation process was complete, resultant nanoparticles were size-filtered using QMF set to select nanoparticles with a size of 3 nm, and then soft-landed on the surface of the silicon substrate in the deposition chamber. All depositions were performed at ambient temperature (about 298 K, as measured by the substrate holder thermocouple). Substrate rotation rate was kept at 2 rpm for all depositions, to ensure best uniformity over the substrate area. No external bias was applied to the substrate, so the landing kinetic energy of the particles was primarily governed by the pressure differential between the aggregation zone and the deposition chamber (the latter typically 2.3×10⁻⁴ mbar during sputtering). Based upon these deposition conditions, landing energy was assumed to be lower than 0.1 eV per atom (Popoka et al. 2011). Surface coverage of tantalum nanoparticles on the substrates was controlled by deposition time. As expected, at low deposition times (5-30 minute) amorphous monodispersed nanoparticles were deposited (referred here as low-coverage samples). For longer deposition times (<60 minute) nanoporous films were obtained (referred here as high-coverage samples, thickness ˜30 nm).

<Analysis>

Samples thus manufactured were evaluated in various ways. AFM (Multimode 8, Bruker, CA) was used for morphological characterization of the deposited nanoparticles. The AFM system height ‘Z’ resolution and noise floor is less than 0.030 nm. AFM scans were performed in tapping mode using commercial silicon-nitride triangular cantilever (spring constant of 0.35 N/m, resonant frequency 65 kHz) tips with a typical radius less than 10 nm. Height distribution curves and rms roughness values were extracted from the AFM images by built-in functions of the scanning probe processor software (SPIP 5.1.8, Image Metrology, Horsholm, DK). Surface topography and nanoparticle size were characterized ex situ, after growth, using SEM (Helios Nanolab 650, FEI Company). TEM studies were carried out using two 300 kV FEI Titan microscopes, equipped with spherical aberration correctors for the probe (for STEM imaging), and the image (for bright field TEM imaging), respectively. In the TEM, energy dispersive x-ray spectrometry (EDS) was performed with an Oxford X-max system, with an 80 mm² silicon drift detector (SDD) and energy resolution of 136 eV. XPS measurements were performed with a Kratos Axis Ultra 39-306 electron spectrometer equipped with a monochromated AlKalpha (1486.6 eV) source operated at 300 W and Ar⁺ ion gun for etching. Spectra/scans were measured at pass energy of 10 eV. The film thickness was evaluated by reflectometry using a NanoCalc thin film reflectometry system (Ocean optics). GIXRD measurements (D8 Discover Bruker CA) were performed using Cu Kₐ radiation (45 kV/40 mA) at a fixed grazing incidence angle of 0.5 degrees.

<Computer Simulation>

Atomistic mechanisms of nanoparticle coalescence were investigated by MD computer simulation, using the Accelrys (copyrighted) Materials Studio Suite. Using the amorphous cell module, nearly spherical amorphous nanoparticles, 3 nm in diameter, were created, with standard room temperature initial density (i.e. 16.69 g/cm³), and containing 792 tantalum atoms. Each created nanoparticle was geometrically optimized and then equilibrated separately for about 50 ps at all temperatures of interest, namely 100, 300, 1000, and 2300 K, using the GULP parallel, classical MD code (Gale 1997) and the embedded-atom method Finnis-Sinclair potential (Finnis and Sinclair 1984). A number of different configurations were subsequently created, combining 2 or 3 nanoparticles of various sizes, and MD runs were performed on them for a production time of 100 ps, using a time-step of 1-3 fs. Nanoparticles were initially brought close to each other, at a separation distance within the potential cut-off radius. Simulations were run at constant temperature, utilizing a Nose-Hoover thermostat with a 0.1 ps parameter. In all cases, the system presented all interesting behavior and reached a stable configuration within the simulation run time.

<Low Coverage: Monodispersed Nanoparticle Deposition>

After the deposition process, a load-lock mechanism allowed samples to be transferred to an adjacent nitrogen gas filled glove-box for characterization, thus avoiding oxidation or contamination. There, surface coverage and size distribution of the as-deposited nanoparticles were studied by AFM. FIG. 3 is (a) AFM topography image of a sample with low tantalum nanoparticle coverage deposited onto a silicon substrate with (b) height histogram. A Gaussian fit to the histogram is shown in the solid line. The average height is 3.8 nm, in good agreement with the pre-selected size of 3.0 nm by the QMF. The sub-monolayer, low-coverage nature of these samples is evident in the soft tapping-mode AFM image shown in FIG. 3(b). As deposition occurred at low kinetic energy, nanoparticles retained their original shapes. Bright spots resulted from aggregates of two or more nanoparticles, probably arising from their “piling up” on the surface. The height distribution (FIG. 3(b)) can be fitted quite well with a Gaussian curve with a peak height (average size) at 3.8 nm. The average size measured by AFM appears is in good agreement with the QMF selected size of 3 nm.

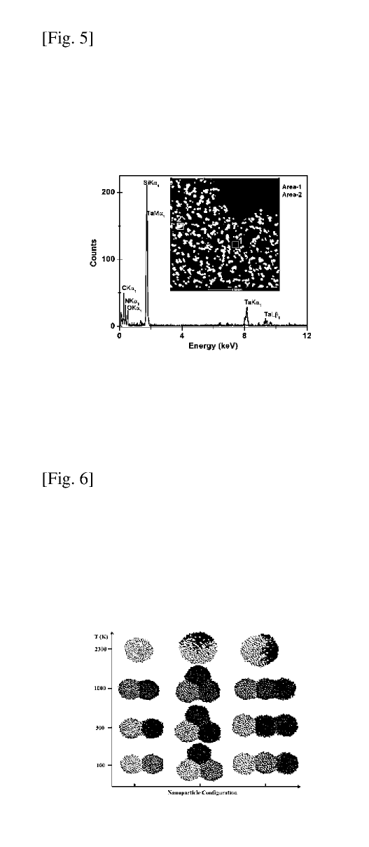

After air exposure, the samples were examined by TEM and HAADF-STEM. FIG. 4 shows (a) Bright-field TEM and (b) high-angle-annular dark-field STEM micrographs of tantalum/tantalum oxide nanoparticles directly deposited on silicon nitride membrane. The insets show high-magnification image, where particles are amorphous in nature (inset in (a)) and a core-shell structure consisting of a metallic tantalum core covered with tantalum oxide (inset in (b)) It was found that the low-coverage tantalum/tantalum oxide nanoparticles had an elongated shape, resulting from coalescence of individual nanoparticles on the Si₃N₄ substrate (TEM grid) surface during deposition (FIGS. 4(a) and 4(b)). In HAADF-STEM, in z-contrast imaging mode, most nanoparticles have a central bright spot within a shell of a slightly lower intensity (see, FIG. 4(b) inset, for example). This suggests a core-shell structure, consistent with a metallic tantalum core covered with tantalum oxide. This tantalum oxide shell is attributed to oxidation of tantalum nanoparticles when exposed to ambient atmosphere. Around a roughly spherical core of amorphous pure tantalum about 3 nm in diameter, an amorphous tantalum oxide shell was formed, with a thickness of about ˜2 nm. FIG. 5 shows the measured EDS spectra of tantalum/tantalum oxide nanoparticles and between nanoparticles. The EDS spectra indicate that the nanoparticle area (marked by numeral 2) contains Ta and O, as expected.

<High Coverage: from Monodispersed Nanoparticles to Porous Films>

For longer deposition times, continuous layers of tantalum nanoparticles were deposited, first on the surface of the silicon substrate and then on each other. Extended coalescence between nanoparticles led to the formation of a porous thin film. To fully understand the nature of the atomistic mechanisms that govern this coalescence, a number of molecular dynamics computer simulations were run. Previously, coalescence has been extensively studied by means of MD for a number of materials such as gold (Lewis et al. 1997; Arcidiacono et al. 2004), silver (Zhao et al. 2001), copper (Kart et al. 2009; Zhu and Averback 1996), iron (Ding et al. 2004), etc. All studies agree, in general terms, on a common mechanism. By sintering together, nanoparticles reduce their free surface area, creating an interface and thus reducing their overall potential energy. After this primary interaction, necks are formed at the interface, assisted by atomic diffusion. These necks are also considered to be the most chemically active sites, the so-called 3-phase boundaries (Eggersdorfer et al. 2012). Their thickness heavily influences the film properties, which depend on porosity, such as mechanical stability, electrical conductivity, and gas sensitivity.

FIG. 6 show examples of characteristic resultant aggregates after a 100 ps molecular dynamics run for two and three nanoparticle configurations at temperature range 100 K to 2300 K for systems consisting of 2 or 3 nanoparticles. Combinations of such aggregates create the structures of nanoporous films developed by nanoparticle deposition (different gray scale combinations represent different temperatures, for clarity). The significance of the effect of temperature is evident for all structures. At 2300 K, near the melting temperature of 3 nm tantalum nanoparticles (2500 K for the potential used), full consolidation into a single, larger nanoparticle occurs in all cases. Temperatures this high cannot be found on (or near) the substrate, but are realistic inflight, as the still-hot nanoparticles may impact one another, within or upon leaving the aggregation zone. At lower temperatures, all configurations present similar, less pronounced, degrees of coalescence. Such behavior corresponds to nanoparticles contacting each other on the substrate at room temperature due to atomic surface diffusion, and sintering together, thus forming an interface that takes the form of a neck. The widths of these necks depend on temperature and determine the final shape and fractal dimension of the aggregates, and, subsequently, the porosity of the resultant film, since it is combinations of aggregates such as those depicted in FIG. 6 that create the structure of the final nanoporous film, as seen in FIG. 4.

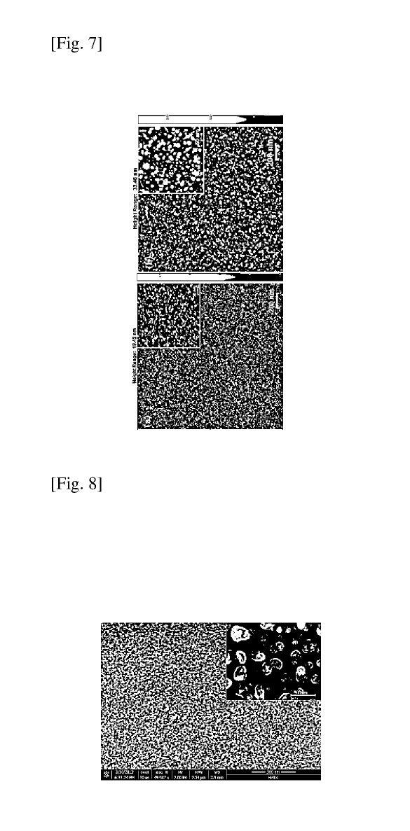

FIG. 7 shows AFM surface morphology of (a) non-oxidized high coverage tantalum nanoparticles deposited onto a silicon substrate and (b) oxidized high coverage tantalum nanoparticles deposited onto a silicon substrate. The respective insets show a high-magnification image, which shows that the roughness increased after oxidation of Ta nanoparticles. FIG. 7 shows that the quality of the films is very good, and more importantly, they are porous. It is shown that when the high-coverage Ta nanoparticles film is exposed to air, an oxide layer is formed at its surface with an associated increase in the measured rms roughness from 2.12 nm to 2.86 nm. Further, as shown in FIG. 8, the porous nature of the film can be verified by SEM after air-exposure, where extended oxidation led to a continuous, layered structure. Tantalum nanoparticles were homogeneous in size, and closely stacked on each other. The inset in FIG. 8 shows both near-spherical and elongated nanoparticle aggregates, similar in shape to the simulated ones (FIG. 6). The fine substructure is due to the small average size of the original nanoparticles (3-4 nm). Pores were created as nanoparticles land on random sites, either on the substrate or on nanoparticles of a lower layer, and their sizes are comparable to those of the nanoparticles. However, their openings, i.e., the top layer of pores, are typically much larger than the cross-sectional area of the nanoparticles. Therefore, as new nanoparticles were deposited, they easily penetrated the uppermost pore layers, until they finally landed, partially coalescing with previously deposited nanoparticles. This caused the lower layers of the film to develop a denser structure than the ones near the surface.

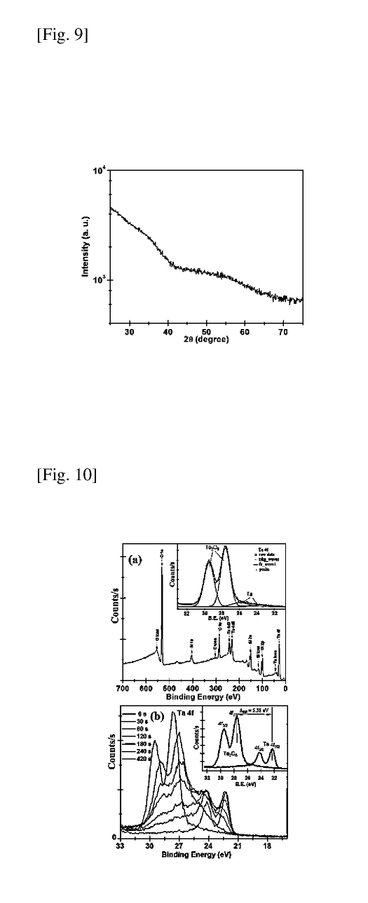

FIG. 9 shows the observed grazing angle X-ray diffraction pattern of a nanoporous film on a silicon substrate at a fixed grazing angle of 0.5°. No peaks given for tantalum and tantalum oxide phases can be observed, except for a broad diffuse peak, which is typically the signature of amorphous nanoparticle films. Thus, the amorphous state of the film was confirmed by GIXRD measurements shown in FIG. 9. No peaks associated with crystalline tantalum and tantalum oxide phases were detected, while from a broad diffuse peak, typical of amorphous nanoparticle films was detected (Stella et al. 2009).

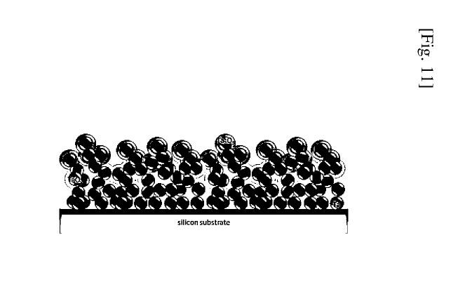

Finally, the qualitative chemical composition and bonding states of the obtained nanoporous film were characterized by XPS. FIG. 10 shows an XPS survey spectrum of high coverage nanoporous film deposited on a silicon substrate. Signals from Ta 4f, Ta 2p, Si 2p, Si 2s, and O 1s edges were observed in the XPS analysis. The deposited Ta nanoparticle film is highly oxidized due to air exposure. Here, metallic (tantalum) formed a variety of oxides such as Ta₂O₅ (the predominant, most stable phase) and suboxides (TaO and TaO₂ which are generally metastable phases) (Hollaway and Nelson 1979; Kerrec et al. 1998; Chang et al. 1999; Atanassova et al. 2004; Moo et al. 2013). The inset in FIG. 10(a) shows the Ta 4f core level spectra of the high coverage porous film. At the surface of the film (first levels), Ta 4f doublet (4f_{7/2}, 4f_{5/2}) fitted with peaks located at binding energies of 27.61 eV and 29.49 eV is observed (energy separation of 1.88 eV) (Chang et al. 1999). These binding energies are close to the stoichiometric Ta₂O₅ and suggest that the film is oxidized to the Ta⁵⁺ state. Metallic tantalum is also detected in the low intensity doublet at binding energies of 23.78 and 25.94 eV.

A depth profile experiment was carried out by surface etching (from the surface level to the last etching up to 420 sec) for the high coverage porous film by monitoring the Ta 4f core level (FIG. 10(b)). Ta 4f doublets are observed at the same binding energy as shown previously in this text. After three etching iterations, the intensity of metallic tantalum (Ta⁰) is increased. These data shows a clear doublet (two peaks) at binding energies of 25.94 (4f_{7/2}) and 23.78 (4f_{5/2}) eV (Chang et al. 1999). Moreover, the intensity of Ta⁵⁺ decreases with increasing etching time and recorded spectra show two states, namely Ta⁰ and Ta⁵⁺. The relative proportions change gradually until the peaks corresponding to the Ta⁵⁺ state disappear. The inset spectra of FIG. 10(b) show that the binding energy difference (DE_{BE}) between the peaks (4f_{7/2}) of metallic tantalum and tantalum oxide is 5.38 eV. These results confirm that the oxidation state of Ta is +5 (i.e. Ta₂O₅) at (and near) the surface of the obtained film (Chang et al. 1999; Hollaway and Nelson 1979).

Regarding the apparent graded composition of the film, while preferential sputtering of oxygen has been reported previously, that is not considered significant on our films, given the relatively high accelerating voltage (6 KeV) that was used (Hollaway and Nelson 1979). It is believed that a plausible interpretation of the graded chemical composition of the film can be attributed to the morphology of the film. As explained previously, at the beginning of the deposition process, monodispersed nanoparticles are deposited on the surface of the substrate. By increasing the deposition time, nanoparticles continue to arrive and soft-land on the surface of the substrate, leading to a porous tantalum thin film. Upon exposure of the deposited films to the atmosphere, nanoparticles on, and near, the surface of the film become fully oxidized leading to a homogeneous Ta₂O₅ layer on the surface. Then oxygen from the atmosphere continues progressing through the pores leading to different states of oxidation throughout the film's volume. This is depicted by a schematic illustration shown in FIG. 11. FIG. 11 is a schematic illustration of an example of the porous tantalum film with a graded oxidation profile perpendicular to the surface of the substrate according to the present invention, which have been realized through the studies explained above. Larger pore sizes near the surface allow for oxidation of tantalum to tantalum oxide. Oxidation levels decrease deeper into the film, leading to pure metallic tantalum near the film/substrate interface.

The present inventor also conducted a research to explore application of the disclosed graded-oxide tantalum porous films to dental implants. A dental implant base made of a Ti-alloy was coated by a tantalum oxide nanoparticles film of the present invention. It was found that the dental implant coated with the film of the present invention is superhydrophilic initially, but once exposed to water, becomes hydrophobic, which is very advantageous in dental implant procedures conducted by dentists.

The dental implant base may be made of other materials, such as a tungsten alloy. Furthermore, it is evident from this research, the graded-oxide tantalum porous films of the present invention can be coated on other biomedical implants, such as hip and joint implants, to provide for superior biomedical implants.

Furthermore, a mono-disperse layer of silver (Ag) may be deposited on top of the graded tantalum oxide (TaOx) film of the present invention, which confers antimicrobial properties. The apparatus of the present invention disclosed above can be used to deposit both the TaOx and the mono-disperse Ag nanoparticles, without modification. The anti-microbial properties of Ag itself are well known, and provide additional advantages for the medical, dental and biological applications of the present invention.

The controlled size and spherical defect-free tantalum oxide nanoparticles film disclosed by the present disclosure is applicable to various applications such as porous films for inorganic-TFT or optical coatings. A graded oxidation profile results in different surface properties at the lower and upper interfaces, respectively, and will be useful, for example, in engineering adhesion to different substrates or cellular materials at the upper and lower interfaces. A nanostructured film, in general, offers much greater surface area than a traditional thin film of corresponding thickness and associated benefits for liquid and gas-based applications. Constraining the size and porosity at the nanoscale also allows engineering of tailored optical and electronic properties.

The present disclosure describes the design and assembly of porous films with a graded oxidation profile perpendicular to the substrate surface, using size-selected tantalum nanoparticle deposition. A number of diagnostic methods were utilized for their characterization. Surface morphological analysis by AFM clearly demonstrated the porous structure of the films, governed by nanoparticle coalescence, as indicated by MD simulations. SEM and HRTEM/HAADF-STEM imaging confirmed this structure after air exposure and the resultant oxidation of nanoparticles to core/shell tantalum/tantalum oxide configurations. GIXRD identified nanoparticles as amorphous. XPS analysis demonstrated the graded nature of oxidation. At the top-most layers of the film, the larger free-surface areas of nanoparticles enabled the formation of Ta₂O₅, which is the thermodynamically stable tantalum oxide. At lower layers, smaller pores of the films allowed only partial diffusion of oxygen, leading to less oxidized states. Pure metallic tantalum was detected at the film/substrate interface. Control of this graded oxidation allows for surface manipulation and design of nanoporous films for various biomedical and technological applications.

It will be apparent to those skilled in the art that various modification and variations can be made in the present invention without departing from the spirit or scope of the invention. Thus, it is intended that the present invention cover modifications and variations that come within the scope of the appended claims and their equivalents. In particular, it is explicitly contemplated that any part or whole of any two or more of the embodiments and their modifications described above can be combined and regarded within the scope of the present invention.

### REFERENCE SIGNS LIST

101 Linear Drive

103 Connection for Coolant water

105 Sputter Target Material (Ta)

107 Turbo Pump Port

109 Super-saturated Ta Vapor

111 Aggregation zone (NP beam source)

113 Sample Deposition Chamber

115 Substrate

117 Quadruple Mass Filter (QMF)

119 Aperture

121 DC Magnetron

123 Pressure Gauge

125 Aggregation Gas Feed

127 Connections for DC Power and Gas

1. A porous film made of size-selected tantalum nanoparticles, formed on a substrate, the porous film having a graded oxidation profile perpendicular to a surface of the substrate.

2. The porous film made of size-selected tantalum nanoparticles according to claim 1, wherein oxidation of the tantalum nanoparticles is higher on a top surface of the film and is progressively lower towards a bottom surface of the film that is on the substrate.

3. The porous film made of size-selected tantalum nanoparticles according to claim 1, further comprising a mono-disperse layer of silver (Ag) deposited on the porous film, thereby providing enhanced antimicrobial properties.

4. The porous film made of size-selected tantalum nanoparticles according to claim 2, further comprising a mono-disperse layer of silver (Ag) deposited on the porous film, thereby providing enhanced antimicrobial properties.

5. A dental implant comprising:
an implant base; and
a porous film made of size-selected tantalum nanoparticles, formed on the implant base, the porous film having a graded oxidation profile perpendicular to a surface of the implant base.

6. The dental implant according to claim 5, wherein oxidation of the tantalum nanoparticles in the porous film is higher on a top surface of the film and is progressively lower towards a bottom surface of the film that is on the implant base.

7. The dental implant according to claim 5, further comprising a monodisperse layer of silver (Ag) deposited on the porous film, thereby providing enhanced antimicrobial properties.

8. The dental implant according to claim 6, further comprising a monodisperse layer of silver (Ag) deposited on the porous film, thereby providing enhanced antimicrobial properties.

9. The dental implant according to claim 5, wherein the implant base is made of a Ti alloy.

10. A biomedical implant comprising:
an implant base; and
a porous film made of size-selected tantalum nanoparticles, formed on the implant base, the porous film having a graded oxidation profile perpendicular to a surface of the implant base.
